(19) United States
(12) Patent Application Publication
(10) Pub. No.: US 2009/0275057 A1
(43) Pub. Date: Nov. 5, 2009

(54) Diagnostic markers predictive of outcomes in colorectal cancer treatment and progression and methods of use thereof

(76) Inventors: Steven P. Linke, La Jolla, CA (US); Troy M. Bremer, La Jolla, CA (US); Cornelius A. Diamond, La Jolla, CA (US)

Correspondence Address: William C. Fuess, 10951 Sorrento Valley Road 2G, San Diego, CA, 92121, US

(21) Appl. No.: 11/731,683

(22) Filed: Mar. 29, 2007

Related U.S. Application Data
(60) Provisional application No. 60/787,893, filed on Mar. 31, 2006.

(51) Int. Cl. G01N 33/574

(52) U.S. Cl. 435/7.23

(57) ABSTRACT

Colorectal cancer patients with operable tumors must decide whether to receive adjuvant therapy after surgical resection in order to reduce their chances of recurrence. Current clinical guidelines are crudely based on the stage of the disease, as well as a few other clinicopathologic features. The instant invention integrates data from these clinicopathologic features with data on multiple biomarkers using advanced informatic methods to provide a far more accurate prediction of recurrence than the current guidelines. The instant invention consists of a panel of biomarker assays plus an algorithm into which the scored biomarker data, as well as standard clinicopathologic data, is entered. A tumor sample from an individual patient is submitted for test, and an individualized report is produced with a prognostic score that accurately reflects the patient's risk of recurrence. This helps guide the patient and his/her oncologist in their choice of whether to receive adjuvant treatment. Low-risk patients are spared the unnecessary toxicities associated with cytotoxic treatments, and high-risk patients are given the best chance for a cure, maximizing both life expectancy and quality of life.

### REFERENCE TO RELATED PATENT APPLICATIONS

The present application is descended from, and claims benefit of priority of, U.S. provisional patent application No. 60/787,893, filed Mar. 31, 2006, which application is hereby incorporated by reference in its entirety.

### GOVERNMENT SUPPORT

The present invention was developed under Research Support of the National Institute of Health. The U.S. Government may have certain rights in this invention.

### FIELD OF THE INVENTION

The present invention generally pertains to the prediction of the outcome of adjuvant therapy, and particularly chemotherapy, in the treatment of colorectal cancer based on the presence and quantities of certain protein molecular markers, called biomarkers, present in the treated patients. The present invention also pertains to the prediction of progression of colorectal cancer, e.g. whether or not the patient's tumour is likely to metastasize, based upon cancer based on the presence and quantities of certain protein molecular markers.

The present invention specifically concerns (1) the identification of groups, or “palettes”, of biomarkers particularly useful in combination for enhanced predictive accuracy of patient response to colorectal cancer therapy with chemotherapy, (2) the identification of certain pairs of biomarkers that, in pairwise combination, are or superior predictive accuracy in the particular estimation of percentage disease-specific survival at most usually and particularly, some 30+ months from onset of adjuvant treatment, and, commensurate with the predictive accuracy of these biomarker pairs, (3) the recognition and quantification of the significance of any changes in any patient biomarkers, and particularly in those biomarkers that, taken pairwise, are of superior predictive accuracy in advanced stages of colorectal cancer.

### BACKGROUND OF THE INVENTION

The following discussion of the background of the invention is merely provided to aid the reader in understanding the invention and is not admitted to describe or constitute prior art to the present invention.

Colorectal cancer is second only to lung cancer as the most common cause of cancer death. In the United States, it is estimated that there will be 106,680 new cases of colon cancer and 41,930 new cases of rectal cancer with a total of 55,170 combined deaths in 2006 (Cancer Facts and Figures 2006. Atlanta, Ga., American Cancer Society, 2006). Worldwide, it is estimated that there are nearly a million new cases and half a million deaths per year due to colorectal cancer (Parkin D M, Bray F, Ferlay J, Pisani P: Estimating the world cancer burden: Globocan 2000. Int J Cancer 94:153-6, 2001). Approximate 5-year overall survival (OS) rates for the four main stages of disease are: 80-95% for stage I (T1-2/N0/M0; primary tumor invasion is limited to the submucosa with no regional lymph node or distant metastasis), 60-80% for stage II (T34/N0/M0; primary tumor invasion beyond the submucosa, but still no nodal or distant metastasis), 25-60% for stage III (T1-4/N>0/M0; regional lymph node, but no distant, metastasis), and <10% for stage IV (M>0; distant metastasis) (Zaniboni A, Labianca R: Adjuvant therapy for stage II colon cancer: an elephant in the living room? Ann Oncol 15:1310-8, 2004).

Approximately 21% of patients are diagnosed with stage IV disease, for which surgery is not a curative option, and radiation and chemotherapy are administered mainly for palliation with very small improvements in median survival. For the remaining patients (˜15% stage I, ˜36% stage II, and ˜28% stage III) (Andre T, Sargent D, Tabernero J, O'Connell M, Buyse M, Sobrero A, Misset J L, Boni C, de Gramont A: Current issues in adjuvant treatment of stage II colon cancer. Ann Surg Oncol 13:887-98, 2006), surgical removal of the primary tumor and regional lymph nodes is typical and can be curative without further treatment. However, adjuvant chemotherapy prevents or delays recurrence in many of these patients. Current clinical guidelines, such as those published by the National Cancer Institute, base the choice of adjuvant treatment primarily on the location of the cancer (colon vs. rectum) and the stage of the disease.

Colon Cancer

In colon cancer, adjuvant chemotherapy is recommended for stage III patients, as standard 5-fluorouracil (FU)-based regimens confer a statistically significant >10% absolute 5-year OS improvement (See for instance NIH consensus conference. Adjuvant therapy for patients with colon and rectal cancer. Jama 264:1444-50, 1990). For stage II colon cancer, recent comprehensive meta-analyses were conducted on dozens of published clinical trials by Figueredo et al. and an American Society of Clinical Oncology (ASCO) panel (See for instance Benson A et al: American Society of Clinical Oncology recommendations on adjuvant chemotherapy for stage II colon cancer. J Clin Oncol 22:3408-19, 2004). They concluded that adjuvant chemotherapy does not provide a statistically significant 5-year OS benefit, so they recommended against making chemotherapy standard for all stage II patients. However, it was recognized that there would likely have been a small (˜2-4%) absolute 5-year OS benefit if there were data on enough stage II patients to reach statistical significance, so it was suggested that stage II patients be urged to participate in clinical trials, and that those with certain risk factors would be good candidates for adjuvant chemotherapy (See for instance Benson A Ibid.).

The high-risk clinicopathologic features in stage II colon cancer identified by ASCO include T4 lesions (primary tumor that has adhered to or invaded adjacent organs), obstruction of the bowel by the tumor, poor histological grade, peritumoral lymphovascular involvement, and potential “understaging” due to too few lymph nodes being examined (<13) (See for instance Benson A Ibid.). The National Comprehensive Cancer Network (NCCN) has adopted similar guidelines of observation, clinical trials, or adjuvant chemotherapy, depending on the presence of these risk factors. However, the factors are relatively crude, <20% of newly diagnosed stage II patients have them (See for instance Midgley R, Kerr D J: Adjuvant chemotherapy for stage II colorectal cancer: the time is right! Nat Clin Pract Oncol 2:364-9, 2005), and it is not necessarily the subset of stage II patients with these risk factors that is benefiting from adjuvant chemotherapy (See for instance Kohne C H: Should adjuvant chemotherapy become standard treatment for patients with stage II colon cancer? Arguing against the proposal is Lancet Oncol 7:516-7, 2006). Emerging evidence indicates that stage II cancers are quite heterogenous, so molecular markers will be critical for the accurate identification of individual high-risk patients as candidates for systemic adjuvant therapy.

Rectal Cancer

In rectal cancer, both adjuvant radiotherapy and chemotherapy (FU-based) have proven beneficial in both stage II and III patients (see for instance Krook J et al.: Effective surgical adjuvant therapy for high-risk rectal carcinoma. N Engl J Med 324:709-15, 1991). Survival of rectal cancer patients is somewhat lower than colon cancer patients in the respective stages of disease. Due to physical constraints in the rectum, surgical margins tend to be much smaller than in colon cancer, so local recurrence is a greater problem, and the quality of the surgery can be a significant prognostic factor (see for instance McArdle C: ABC of colorectal cancer: effectiveness of follow up. Bmj 321:1332-5, 2000). Thus, the benefit of local radiotherapy in rectal vs. colon cancer likely derives from the need to reduce these local recurrences. In addition, prognostic molecular markers are typically common to both colon and rectal cancers, and rectal tissue is more closely related to the left colon, cancers in which tend to have worse prognoses than right colon cancers (see for instance Eisenberg B et al. Carcinoma of the colon and rectum: the natural history reviewed in 1704 patients. Cancer 49:1131-4, 1982). Thus, the benefit of chemotherapy in stage II rectal vs. colon cancer may derive from the need to reduce both local and distant recurrence based on these higher risks. Although chemotherapy is currently recommended for all stage II rectal patients, it has been suggested that surgery alone would be sufficient in a significant fraction of properly selected low-risk patients (see for instance Gunderson L et al. Impact of T and N stage and treatment on survival and relapse in adjuvant rectal cancer: a pooled analysis. J Clin Oncol 22:1785-96, 2004). Given the above observations, a single molecular model to distinguish low- and high-risk patients will likely apply to both types, perhaps with some additional risk of local recurrence in rectal cancer patients.

Current State of the Art

Two online tools, The Mayo Clinic's “Adjuvant Systemic Therapy for Resected Colon Cancer Health Tool” (http://www.mayoclinic.com/calcs in 2007) and Adjuvant! Inc.'s “Adjuvant! Online Colon Cancer” (http://www.adjuvantonline.com/ in 2007), project 5-year disease-free survival (DFS) and OS for stage I-III colon cancer patients, and adjuvant chemotherapy benefit in stage II and III patients. These tools are based on pooled analyses of standard data (patient age/sex, T stage, N stage, and tumor grade) and outcomes from various clinical trials, as well as the SEER Public Registry and the National Cancer Database. Although these tools provide valuable information, they lump patients into relatively broad categories and, thus, have limited discriminative ability. Inclusion of molecular markers will provide a far more accurate, individualized, assessment of risk.

Although a number of studies have been published indicating that one or more markers have or likely have prognostic significance, some studies have not confirmed the findings, and no consensus has been reached on their utility. More importantly; however, the present invention will be seen to demonstrate the importance of the conditional interpretation of certain markers on others due to their interdependency. Some of these studies will be detailed in later sections of the instant invention.

For instance, A number of microarray studies have been published comparing mRNA expression levels of thousands of genes in various tissue combinations, including tumor vs. normal, primary vs. metastases, and different stages of disease (Shih W, Chetty R, Tsao M S: Expression profiling by microarrays in colorectal cancer (Review). Oncol Rep 13:517-24, 2005). Reports of prognostic tests based on gene expression technology have been more limited. In one study of stage II colon cancer patients, a primary analysis method resulted in a 60-gene signature that had zero discriminative ability. An alternative method was then employed using hierarchical clustering to first divide patients into two groups, and then training separately on those groups. Using this method, a 23-gene prognostic signature was developed that purportedly predicted recurrence with 78% accuracy in a set-aside test set of patients (See Wang Y et al. Gene expression profiles and molecular markers to predict recurrence of Dukes' B colon cancer. J Clin Oncol 22:1564-71, 2004). However, the 23-genes were identified from 17,616 total transcripts from two different training sets of only 27 patients (21 disease-free, 6 relapses) and II patients (4 disease-free, 7 relapses), each. The method used to account for multiple comparisons was substantially inadequate to correct for the high expected false discovery rate. In addition, the test set was only 36 patients (18 disease-free, 18 relapses), resulting in very large confidence intervals. Thus there is a very high likelihood that this model is extremely overfit, such that it will not generalize. To our knowledge, there has been no validation of this model since it was published in 2004.

In another gene expression study (See O'Connell M et al. Relationship between tumor gene expression and recurrence in stage II/III colon cancer: Quantitative RT-PCR assay of 757 genes in fixed paraffin-embedded (FPE) tissue. Journal of Clinical Oncology, ASCO Annual Meeting Proceedings Part 124:3518, 2006), 757 genes were studied by RTPCR in 270 stage II and III colon cancer patients, and 148 candidate genes were identified as associated with recurrence-free interval. However, the actual performance of this set of genes is unknown and no effort was made to account for multiple comparisons, other than the authors stating that 25% of the genes are expected to be false positives based on false discovery rate calculations.

A variety of serious questions have been raised by experts about the experimental and statistical methodologies used in gene expression studies, such as the false discovery rate and overfitting issues described above (see for example Ransohoff D F: Rules of evidence for cancer molecular-marker discovery and validation. Nat Rev Cancer 4:309-14, 2004; Simon R: Development and validation of therapeutically relevant multi-gene biomarker classifiers. J Natl Cancer Inst 97:866-7, 2005; Ein-Dor L, Kela I, Getz G. Givol D, Domany E: Outcome signature genes in breast cancer: is there a unique set? Bioinformatics 21:171-8, 2005). Beyond the statistical problems, the analysis methods typically used do not account for complex data distributions or non-linear interactions between genes. In addition, the use of gene expression data is not ideal. For example, microarray data is not at all quantitative, and although RT-PCR can be relatively quantitative, the source material can compromise this (RNA is extracted from homogenized tumor tissue that ranges from ˜50-100% tumor cellularity with ˜0-50% non-tumor tissue—normal epithelial and stromal cells). Also, complex post-translational regulation of many genes is critical for the function of the protein (e.g., stability, subcellular localization, activation, etc.). Unfortunately, simply measuring transcript levels does not account for any of these important issues. In contrast, the instant invention will be seen to use robust, powerful methods to prevent overfitting (maximizing the potential for generalizability) and to discover complex interactions between markers (maximizing performance). In addition, the instant invention will be seen to use protein markers in tumor sections that allow direct, quantitative (and semiquantitative) assessment of the most relevant targets with the ability to discriminate between tumor and nontumor cells, as well as sub-cellular localization. As an added bonus, this type of technology is widely accepted and validated in the pathology community, unlike gene expression approaches.

Other research relevant to colon cancer progression includes U.S. patent application Ser. No. 10/033,570 concerning the association of expression levels of a group of genes, respectively, to chemotherapeutic response. The group of markers was discovered using a support vector machine. As well as suffering from the methods of using gene expression as diagnostic and/or prognostic biomarkers described above, the result has not been replicated since the initial disclosure. U.S. patent application Ser. No. 11/102,228 details several multi-marker panels that predict patient response to chemotherapy based upon “ . . . determining the expression level of one or more prognostic RNA transcripts or their expression products in a biological sample comprising cancer cells obtained from said subject . . . ” and moreover said gene panels are associated with response to chemotherapy in “breast cancer, ovarian cancer, gastric cancer, colon cancer, pancreatic cancer, prostate cancer and lung cancer.” Not only does this described method suffer from the pitfalls of using gene expression as a treatment outcome diagnostic, but all cancers are individual diseases with separate disease processes. This means that the biological response to treatment will differ between different cancers, and therefore any gene expression pattern associated with response to treatment in one cancer will be useless as a signature for similar prognostical ability in a cancer originating from a different tissue type or part of the body. However, as the instant invention describes below and in FIG. 11, the scheme detailed in U.S. patent application Ser. No. 11/061,067 is not enough to produce a diagnostic of sufficient sensitivity and specificity. Still other relevant literature to the instant invention include U.S. patent application Ser. Nos. 10/872,063, 10/883,303, and 10/852,797, which claim gene-expression tests for predicting breast cancer progression and treatment to various chemotherapies. Some of the U.S. patent applications disclosed previously make mention of the protein products of such genes in producing such a test, but (1) this is not enabled in these specific patents and the instant invention is the first to the best of our knowledge to describe a specific set of proteins whose values are interpolated by a specific algorithm to produce a prediction of response to therapy and/or risk of progression; while (2) the instant invention enables in its claims a minimal set of specific protein product biomarkers interpolated by a specific nonlinear algorithms which permit a highly sensitive and specific test validated by independent testing patient populations; while (3) none of these applications details usage of clinicopathological variables as part of determining a clinical outcome in conjunction with molecular markers, as does the instant invention.

In contrast, the present invention will be seen to concern the development of a multi-molecular marker diagnostic with significant contributions by APAF1/CED4, BAG1, BAX, BCL-2, BIRC2/cIAP1, BIRC3/cIAP2, BIRC5/survivin, CARD8/TUCAN, MKI67/MIB1, TP-53, and others, in addition to standard clinicopathological factors such as micro-satellite instability, all interpolated by an algorithm that can deliver superior prognostic ability as compared to individual protein markers or gene expression techniques.

### BRIEF SUMMARY OF THE INVENTION

Provided in the present invention is a method of providing a prognosis of disease-free survival in a cancer patient comprising the steps of obtaining a sample from the patient; and determining various polypeptide levels (e.g. molecular markers) in the sample, wherein change in various polypeptide levels as compared to a control sample indicates the good prognosis of a prolonged disease-free survival. The present invention contemplates a multiple molecular marker diagnostic, the values of each assayed marker collectively interpolated by a non-linear algorithm, to (1) predict the outcomes of adjuvant therapy, particularly chemotherapy, for colorectal cancer in consideration of multiple molecular makers, called biomarkers, of a patient; and (2) identifying whether or not a tumour from a patient is likely to be more aggressive, or malignant, than another, and thus requiring neoadjuvant chemotherapy in addition to surgical and radiological treatment. The model was built by multivariate mathematical analysis of (1) many more multiple molecular markers, called biomarkers, than ultimately proved to be significant in combination for forecasting treatment outcomes, in consideration of (2) real-world, clinical, outcomes of real patients who possessed these biomarkers.

The diagnostic is subject to updating, or revision, as any of (1) new biomarkers are considered, (2) new patient data (including as may come from patients who had their own treatment outcomes predicted) becomes available, and/or (3) new (drug) therapies are administered, all without destroying the validity of the instant invention and of discoveries made during the building, and the exercise, thereof, as hereinafter discussed.

A number of different insights are derived from (1) the building and (2) the exercise of the diagnostic. A primary insight may be considered to be the identification of a number, or “palette”, of biomarkers that are in combination of superior, and even greatly superior, accuracy for predicting the outcomes of for colorectal cancer than would be any one, or even two, markers taken alone. The predictive power of this combination over that of a simple voting panel response is increased by use of an algorithm that interpolates the linear and non-linear collective contributions of said collection to predict the clinical outcome of interest.

A secondary insight from the diagnostic is that certain biomarkers are or increased predictive accuracy of, in particular, percentage disease-specific survival at 30+ months from onset of treatment when these biomarkers taken in pairs. This does not mean that these biomarker pairs are of overall predictive accuracy to the palette of predictive biomarkers. It only means that, when considered in pairs, certain biomarkers provide useful subordinate predictions.

Finally, a tertiary insight that falls out from the identification of biomarker pairs having superior predictive accuracy is that expected disease-specific survival can, and does, vary greatly when, sometimes, but one single one of these biomarkers changes, as during the course of the treatment of single patient.

Theory of the Invention

In accordance with the present invention, exercise of the diagnostic primarily serves to (1) identify pairs of biomarkers that are unusually strongly related, suggesting in these identified pairs avenues for further investigation of disease pathology, and of drugs; and (2) identify and quantify a palette of biomarkers interpolated by a non-linear algorithm having superior predictive capability for prognosis of chemotherapy benefit in colorectal cancer.

In another of its aspects, the instant invention is embodied in methods for choosing one or more marker(s) for diagnosis, prognosis, or therapeutic treatment of colorectal cancer in a patient that together, and as a group, have maximal sensitivity, specificity, and predictive power. Said maximal sensitivity, specificity, and predictive power is in particular realized by choosing one or more markers as constitute a group by a process of plotting receiver operator characteristic (ROC) curves for (1) the sensitivity of a particular combination of markers versus (2) specificity for said combination at various cutoff threshold levels. In addition, the instant invention further discloses methods to interpolate the nonlinear correlative effects of one or more markers chosen by any methodology to such that the interaction between markers of said combination of one or more markers promotes maximal sensitivity, specificity, and predictive accuracy in the diagnosis, prognosis, or therapeutic benefit and treatment of colorectal cancer.

In various aspects, the present invention relates to (1) materials and procedures for identifying markers that are associated with the diagnosis, prognosis, or differentiation of colorectal cancer in a patient; (2) using such markers in diagnosing and treating a patient and/or monitoring the course of a treatment regimen; (3) using such markers to identify subjects at risk for one or more adverse outcomes related to colorectal cancer; and (4) using at one of such markers an outcome marker for screening compounds and pharmaceutical compositions that might provide a benefit in treating or preventing such conditions.

The first three aspects of the present invention are discussed in the following sections below.

A Palette of Biomarkers Relevant to the Prognosis of Outcome in Chemotherapy of Colorectal Cancer

A diagnostic assay relating diverse biomarkers to real-world, clinical, outcomes from chemotherapy of colorectal cancer having being built, optimised and exercised by the present invention as hereinafter explained, a specific palette of molecular markers, also called biomarkers, useful in predicting outcomes to chemotherapy in the treatment of colorectal cancer patients has been identified.

The preferred predictive palette was derived from a multivariate mathematical model where over 20 biomarkers were taken into consideration, and where eight (8) such biomarkers were found to be of improved prognostic significance taken in combination. Specifically, the most preferred palette of biomarkers predictive of outcome in chemotherapy for colorectal cancer include APAF1/CED4, BAG1, BIRC2/cIAP1, BIRC3/cIAP2, BIRC4/XIAP, CARD8/TUCAN, MKI-67/MIB1, and TP-53.

Use of an algorithm in combining the effects of several markers to predict response to therapy.

Provided in the present invention is a method of providing a treatment decision for a colorectal cancer patient on whether or not to receive chemotherapy comprising obtaining a sample from the patient; and determining various molecular marker levels of interest in the sample, inputting such values into an algorithm which has previously correlated in a machine-learning fashion relationships between said molecular marker levels and clinical outcome, wherein output from such an algorithm indicates that cancer is chemotherapy resistant.

Thus, in certain embodiments of the methods of the present invention, a plurality of markers and clinicopathological factors are combined using an algorithm to increase the predictive value of the analysis in comparison to that obtained from the markers taken individually or in smaller groups. Most preferably, one or more markers for adhesion, angiogenesis, apoptosis, catenin, catenin/cadherin proliferation/differentiation, cell cycle, cell-cell interactions, cell-cell movement, cell-cell recognition, cell-cell signalling, cell surface, centrosomal, cytoskeletal, growth factors, growth factor receptors, invasion, metastasis, membrane/integrin, oncogenes, proliferation, tumour suppression, signal transduction, surface antigen, transcription factors and specific and non-specific markers of colorectal cancer are combined in a single assay to enhance the predictive value of the described methods. This assay is usefully predictive of multiple outcomes, for instance: diagnosis of colorectal cancer, then predicting colorectal cancer prognosis, then further predicting response to treatment outcome. Moreover, different marker combinations in the assay may be used for different indications. Correspondingly, different algorithms interpret the marker levels as indicated on the same assay for different indications.

In preferred embodiments, particular thresholds for one or more molecular markers in a panel are not relied upon to determine if a profile of marker levels obtained from a subject are indicative of a particular diagnosis/prognosis. Rather, in accordance with the present invention, an evaluation of the entire profile is made by (1) first training an algorithm with marker information from samples from a test population and a disease population to which the clinical outcome of interest has occurred to determine weighting factors for each marker, and (2) then evaluating that result on a previously unseen population. Certain persons skilled in bioinformatics will recognise this procedure to be tantamount to the construction, and to the training, of a neural network. The evaluation is determined by maximising the numerical area under the ROC curve for the sensitivity of a particular panel of markers versus specificity for said panel at various individual marker levels. From this number, the skilled artisan can then predict a probability that a subject's current marker levels in said combination is indicative of the clinical marker of interest. For example, (1) the test population might consist solely of samples from a group of subjects who have had Dukes stage B colon cancer and had a recurrence event, while (2) the disease population might consist solely of samples from a group of subjects who have had Dukes stage B colon cancer and did not have a recurrence event. A third, “normal” population might also be used to establish baseline levels of markers as well in a non-diseased population.

In preferred embodiments of the marker, and marker panel, selection methods of the present invention, the aforementioned weighting factors are multiplicative of marker levels in a non-linear fashion. Each weighting factor is a function of other marker levels in the panel combination, and consists of terms that relate individual contributions, or independent and correlative, or dependent, terms. In the case of a marker having no interaction with other markers in regards to then clinical outcome of interest, then the specific value of the dependent terms would be zero.

### Other Embodiments of the Instant Invention

In another embodiment of the instant invention, the response to therapy is a complete pathological response.

In a preferred embodiment, the subject is a human patient.

If the tumor is colorectal cancer, it can, for example, be Dukes stage A, B, C, or D colorectal cancer.

In one specific embodiment of the invention, the patient is not receiving an chemotherapy, a platinum-based therapy or a targeted therapy. In another embodiment, the patient is concurrently receiving a chemotherapy, and a targeted therapy or radiation therapy. In a specific embodiment, the chemotherapy comprises Irinotecan, Leucovorin, and/or 5-Fluorouracil. In a further specific embodiment, the platinum therapy is satraplatin, carboplatin, or cisplatin. In a further specific embodiment, the targeted therapy is bevacizumab, panitumumab, or cetuximab.

In a particular embodiment, the adjuvant treatment is adjuvant chemotherapy.

In another embodiment, the adjuvant treatment is a platinum-based therapy.

The method may involve determination of the expression levels of at least two, or at least three, or at least four, or at least 5, or at least 6, or at least 7, or at least 8, or at least 9, or at least 10, or at least 15, or at least 20 of the prognostic proteins listed within this specification, listed above, or their associative protein expression products.

The biological sample may be e.g. a tissue sample comprising cancer cells, where the tissue can be fixed, paraffin-embedded, or fresh, or frozen.

In a particular embodiment, the tissue is from any type of biopsy.

The expression level of said prognostic protein levels or associated protein levels can be determined, for example, by immunohistochemistry or a western blot, or a fluorescence-based assay, or other proteomics techniques, or any other methods known in the art, or their combination.

In an embodiment, the assay for the measurement of said prognostic proteins or their associated expression products is provided is provided in the form of a kit or kits for staining of individual proteins upon sections of tumor tissue.

In another embodiment, said kit is designed to work on an automated platform for analysis of cells and tissues such as described in U.S. patent application Ser. No. 10/062308 entitled ‘Systems and methods for automated analysis of cells and tissues’.

An embodiment of the invention is a method of screening for a compound that improves the effectiveness of an chemotherapy in a patient comprising the steps of: introducing to a cell a test agent, wherein the cell comprises polynucleotide(s) mentioned in the instant invention encoding polypeptide(s) under control of a promoter operable in the cell; and measuring said polypeptide level(s), wherein when the level(s) are decreased following the introduction, the test agent is the compound that improves effectiveness of the chemotherapy in the patient. It is also contemplated that such an agent will prevent the development of chemotherapy resistance in a patient receiving such a therapy. In a specific embodiment, the patient is chemotherapy-resistant. In a further specific embodiment, the chemotherapy comprises an adjuvant. It is also contemplated that the compound is a ribozyme, an antisense nucleotide, a receptor blocking antibody, a small molecule inhibitor, or a promoter inhibitor.

An embodiment of the invention is a method of screening for a compound that improves the effectiveness of an chemotherapy in a patient comprising the steps of: contacting a test agent with polypeptide(s) mentioned in the instant invention, wherein said polypeptide(s) or the ER polypeptide is linked to a marker; and determining the ability of the test agent to interfere with the binding of said polypeptide(s), wherein when the marker level(s) are decreased following the contacting, the test agent is the compound that improves effectiveness of the chemotherapy in the patient. In certain embodiments of the invention, the patient is chemotherapy-resistant.

One embodiment of the invention is as a method of treating a cancer patient comprising administering to the patient a therapeutically effective amount of an antagonist of polypeptide(s) (as discussed hereinafter) and an chemotherapy. In certain embodiments of the invention, the patient is chemotherapy-resistant. A specific embodiment of the invention is presented wherein the antagonist interferes with translation of the polypeptide(s) (as discussed hereinafter). In a further specific embodiment of the invention the antagonist interferes with an interaction between the polypeptide(s) (as discussed hereinafter) and an estrogen receptor polypeptide. The antagonist interferes with phosphorylation or any other posttranslational modification of the said polypeptide(s). In yet another specific embodiment of the invention the antagonist inhibits the function of a polypeptide encoding a kinase that specifically phosphorylates said polypeptide(s). In yet another embodiment, the antagonist is administered before, together with, or after the chemotherapy. The antagonist and the chemotherapy are administered at the same time in another embodiment.

Another embodiment of the invention is as a method of improving the effectiveness of an chemotherapy in a cancer patient comprising administering a therapeutically effective amount of an antagonist of polypeptide level(s) (as discussed hereinafter) to the patient to provide a therapeutic benefit to the patient. In a specific embodiment, the administering is systemic, regional, local or direct with respect to the cancer.

Another embodiment of the invention is as a method of treating a cancer patient comprising: identifying an antagonist of polypeptide(s) mentioned in the instant invention by introducing to a cell a test agent, wherein the cell comprises a polynucleotide encoding a polypeptide(s) (as discussed hereinafter) under control of a promoter operable in the cell, and measuring the AIB1 polypeptide level. This transpires wherein when the level is decreased following the introduction, the test agent is the antagonist of the said polypeptide(s). Finally transpiring is d administration to the patient a therapeutically effective amount of the antagonist. In certain embodiments of the invention, the patient is chemotherapy-resistant.

Other embodiments, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows is a Table of immunohistochemical markers studied and their feature classification in the prognostic model.

FIG. 2 is comprised of three Kaplan-Meier survival curves for the untreated patient cohort for patients who had (A) either colon or rectal cancer; (B) colon cancer; and © rectal cancer.

FIG. 3 is comprised of three Kaplan-Meier survival curves for the 5-FU treated patient cohort for patients who had (A) either colon or rectal cancer; (B) colon cancer; and © rectal cancer.

FIG. 4 is Table 4 of patient characteristics.

FIG. 5 is a Table Multivariate Cox Proportional hazard analysis of the patient cohorts for three-year survival.

FIG. 6 is a receiver-operator curve plotting the sensitivity of the prognostic model versus 1-specificity, or false positive rate.

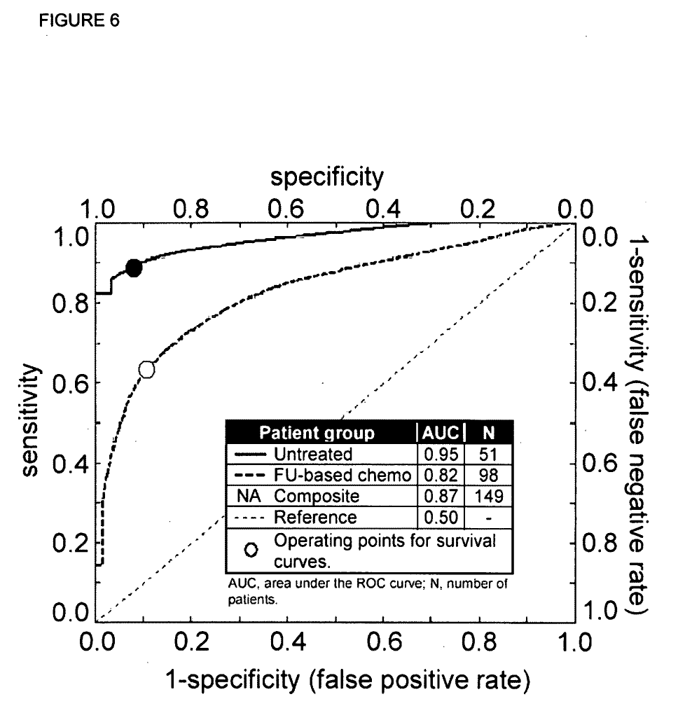

### DETAILED DESCRIPTION OF THE INVENTION

### Definitions

As used herein, the term “adjuvant” refers to a pharmacological agent that is provided to a patient as an additional therapy to the primary treatment of a disease or condition.

The term “algorithm” as used herein refers to a mathematical formula that provides a relationship between two or more quantities. Such a formula may be linear or non-linear, and may exist as various numerical weighting factors in computer memory.

The term “interaction of two or more markers” refers to an interaction that is functional or productive. Such an interaction may lead to downstream signaling events. Other contemplated interactions allow further productive binding events with other molecules.

The term “control sample” as used herein indicates a sample that is compared to a patient sample. A control sample may be obtained from the same tissue that the patient sample is taken from. However, a noncancerous area may be chosen to reflect the individual polypeptide levels in normal cells for a particular patient. A control may be a cell line, such as MCF-7, in which serial dilutions are undertaken to determine the exact concentration of elevated polypeptide levels. Such levels are compared with a patient sample. A “control sample” may comprise a theoretical patient with an elevated polypeptide level of a certain molecule that is calculated to be the cutoff point for elevated polypeptide levels of said certain molecule. A patient sample that has polypeptide levels equal to or greater than such a control sample is said to have elevated polypeptide levels.

As used herein, the term “overall survival” is defined to be survival after first diagnosis and death. For instance, long-term overall survival is for at least 5 years, more preferably for at least 8 years, most preferably for at least 10 years following surgery or other treatment.

The term “disease-free survival” as used herein is defined as a time between the first diagnosis and/or first surgery to treat a cancer patient and a first reoccurrence. For example, a disease-free survival is “low” if the cancer patient has a first reoccurrence within five years after tumor resection, and more specifically, if the cancer patient has less than about 55% disease-free survival over 5 years. For example, a high disease-free survival refers to at least about 55% disease-free survival over 5 years.

The term “Dukes' stage x” where x is A, B, C, or D as used herein refers to the current, non-molecular-marker based classification of colorectal cancer. Survival of patients with colon and/or rectal cancer depends to a large extent on the stage of the disease at diagnosis. Devised nearly seventy years ago, the modified Dukes' staging system for colon cancer, discriminates four stages (A, B, C, and D), primarily based on clinicopathologic features such as the presence or absence of lymph node or distant metastases. Specifically, colonic tumors are classified by four Dukes' stages: A, tumor within the intestinal mucosa; B, tumor into muscularis mucosa; C, metastasis to lymph nodes and D, metastasis to other tissues. Of the systems available, the Dukes' staging system, based on the pathological spread of disease through the bowel wall, to lymph nodes, and to distant organ sites such as the liver, has remained the most popular. Despite providing only a relative estimate for cure for any individual patient, the Dukes' staging system remains the standard for predicting colon cancer prognosis, and is the primary means for directing adjuvant therapy.

The term “chemotherapy-resistant patient” as used herein is defined as a patient receiving an chemotherapy and lacks demonstration of a desired physiological effect, such as a therapeutic benefit, from the administration of an chemotherapy.

The term “polypeptide” as used herein is used interchangeably with the term “protein”, and is defined as a molecule which comprises more than one amino acid subunits. The polypeptide may be an entire protein or it may be a fragment of a protein, such as a peptide or an oligopeptide. The polypeptide may also comprise alterations to the amino acid subunits, such as methylation or acetylation. The term “molecular marker” or biomarker is also used interchangeably with the terms protein and polypeptide, though the two latter terms are subclasses of the former.

The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “prognosis” as used herein are defined as a prediction of a probable course and/or outcome of a disease. For example, in the present invention the combination of several protein levels together with an interpolative algorithm constitutes a prognostic model for resistance to chemotherapy in a cancer patient.

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

The term “therapeutic benefit” as used herein refers to anything that promotes or enhances the well-being of the subject with respect to the medical treatment of his condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition. In a specific embodiment, a therapeutic benefit refers to reversing de novo chemotherapy-resistance or preventing the patient from acquiring an chemotherapy-resistance.

The term “treatment” as used herein is defined as the management of a patient through medical or surgical means. The treatment improves or alleviates at least one symptom of a medical condition or disease and is not required to provide a cure. The term “treatment outcome” as used herein is the physical effect upon the patient of the treatment.

The term “sample” as used herein indicates a patient sample containing at least one tumor cell. Tissue or cell samples can be removed from almost any part of the body. The most appropriate method for obtaining a sample depends on the type of cancer that is suspected or diagnosed. Biopsy methods include needle, endoscopic, and excisional. The treatment of the tumor sample after removal from the body depends on the type of detection method that will be employed for determining individual protein levels.

### DETAILED DESCRIPTION OF THE INVENTION

Most existing statistical and computational methods for biomarker identification of disease states, disease prognosis, or treatment outcome, such as U.S. patent application Ser. Nos. 11/102,228 and/or U.S. patent application Ser. No. 10/210,314, have focused on differential expression of markers between diseased and control data sets. This metric is tested by simple calculation of fold changes, by t-test, and/or F test. These are based on variations of linear discriminant analysis (i.e., calculating some or the entire covariance matrix between features).

However, the majority of these data analysis methods are not effective for biomarker identification and disease diagnosis for the following reasons. First, although the calculation of fold changes or t-test and F-test can identify highly differentially expressed biomarkers, the classification accuracy of identified biomarkers by these methods, is, in general, not very high. This is because linear transforms typically extract information from only the second-order correlations in the data (the covariance matrix) and ignore higher-order correlations in the data. We have shown that proteomic datasets are inherently non-symmetric (See for Instance Linke et al. Clin. Can. Research Feb. 15, 2006). For such cases, non-linear transforms are necessary. Second, most scoring methods do not use classification accuracy to measure a biomarker's ability to discriminate between classes. Therefore, biomarkers that are ranked according to these scores may not achieve the highest classification accuracy among biomarkers in the experiments. Even if some scoring methods, which are based on classification methods, are able to identify biomarkers with high classification accuracy among all biomarkers in the experiments, the classification accuracy of a single marker cannot achieve the required accuracy in clinical diagnosis. Third, a simple combination of highly ranked markers according to their scores or discrimination ability is usually not be efficient for classification, as shown in the instant invention. If there is high mutual correlation between markers, then complexity increases without much gain.

Accordingly, the instant invention provides a methodology that can be used for biomarker feature selection and classification, and is applied in the instant application to prognosis of colorectal cancer and adjuvant treatment outcome.

Exemplary Biomarkers related to prognosis of colorectal cancer and adjuvant treatment outcome.

A comprehensive methodology for identification of one or more markers for the prognosis, diagnosis, and detection of disease has been described previously. Suitable methods for identifying such diagnostic, prognostic, or disease-detecting markers are described in detail in U.S. Pat. No. 6,658,396, NEURAL NETWORK DRUG DOSAGE ESTIMATION, U.S. patent application Ser. No. 09/611,220, entitled NEURAL-NETWORK-BASED INDENTIFICATION, AND APPLICATION, OF GENOMIC INFORMATION PRACTICALLY RELEVANT TO DIVERSE BIOLOGICAL AND SOCIOLOGICAL PROBLEMS, filed Jul. 6, 2000, and U.S. provisional patent application Ser. No. 10/948,834, entitled DIAGNOSTIC MARKERS OF CARDIOVASCULAR ILLNESS AND METHODS OF USE THEREOF, filed Sep. 23, 2003, each of which patents and parent applications is hereby incorporated by reference in its entirety, including all tables, figures, and claims. Briefly, our method of predicting relevant markers given an individual's test sample is an automated technique of constructing an optimal mapping between a given set of input marker data and a given clinical variable of interest.

We first obtain patient test samples of tissue from two or more groups of patients. The patients are those exhibiting symptoms of a disease event, say colorectal cancer, and who are prescribed a specific therapeutic treatment which has a specific clinical outcome are compared to a different set of patients also exhibiting the same disease event but with different therapeutic treatments and/or clinical outcome of said treatment. These second sets of patients are viewed as controls, though these patients might have another disease event distinct from the first. Samples from these patients are taken at various time periods after the event has occurred, and assayed for various markers as described within. Clinicopathological information, such as age, microsatellite instability, T4 lesions, obstruction of the bowel by the tumor, peritumoral lymphovascular involvement, tumor stage, tumor histological grade, and node status are collected at time of diagnosis. These markers and clinicopathological information form a set of examples of clinical inputs and their corresponding outputs, the outputs being the clinical outcome of interest, for instance colorectal cancer prognosis and/or colorectal cancer therapeutic treatment outcome.

We then use an algorithm to select the most relevant clinical inputs that correspond to the outcome for each time period. This process is also known as feature selection. In this process, the minimum number of relevant clinical inputs that are needed to fully differentiate and/or predict disease prognosis, diagnosis, or detection with the highest sensitivity and specificity are selected for each time period. The feature selection is done with an algorithm that selects markers that differentiate between patient disease groups, say those likely to have recurrence versus those likely to no recurrence. The relevant clinical input combinations might change at different time periods, and might be different for different clinical outcomes of interest.

We then train a classifier to map the selected relevant clinical inputs to the outputs. A classifier assigns relative weightings to individual marker values. We note that the construct of a classifier is not crucial to our method. Any mapping procedure between inputs and outputs that produces a measure of goodness of fit, for example, maximizing the area under the receiver operator curve of sensitivity versus 1-specificity, for the training data and maximizes it with a standard optimization routine on a series of validation sets would also suffice.

Once the classifier is trained, it is ready for use by a clinician. The clinician enters the same classifier inputs used during training of the network by assaying the selected markers and collecting relevant clinical information for a new patient, and the trained classifier outputs a maximum likelihood estimator for the value of the output given the inputs for the current patient. The clinician or patient can then act on this value. We note that a straightforward extension of our technique could produce an optimum range of output values given the patient's inputs as well as specific threshold values for inputs.

One versed in the ordinary state of the art knows that many other proteomic biomarkers in the literature once measured from tumor tissue in a diseased patient and healthy tissue from a healthy patient, selected through use of an feature selection algorithm might be prognostic of colorectal cancer or colorectal cancer treatment outcome if measured in combination with others and evaluated together with a nonlinear classification algorithm. We now described some of these other polypeptides, previously considered for diagnosis or prognosis of colorectal cancer and thus not novel in themselves. This list is meant to serve as illustrative and not meant to be exhaustive.

Markers of Colorectal Cancer Progression

A large number of colorectal cancer prognostic markers from several biological pathways have been identified in the literature over the years, most of them based on IHC analysis of tumor sections. Despite all of the research resources expended, none of these individual markers have achieved the level of evidence required for routine usage33. The multi-marker strategy proposed here is most likely to succeed if markers are chosen that have the highest level of evidence of prognostic power from a broad range of biological pathways. Further, we propose to study additional markers related to cell cycle, cell adhesion, metastasis, invasion, angiogenesis, nucleotide biosynthesis, and receptor-based growth/death signaling (Table 7). The following background information on some of these markers (identified by bold text) contains citations for studies that show or summarize prognostic significance by IHC. Virtually all of these studies suggest the usage of the presented marker(s) in the future prognosis and/or treatment decisions for colorectal cancers.

Angiogenesis, or formation of new blood vessels, is important for both tumor growth and development of metastases. VEGF is a glycoprotein related to platelet-derived growth factor that is considered the primary stimulator of angiogenesis. Several studies have shown that tumor VEGF expression is a significant negative prognostic factor in colorectal cancer patients (See for instance Maeda K et al. Expression of vascular endothelial growth factor and thrombospondin-1 in colorectal carcinoma. Int J Mol Med 5:373-8, 2000)

Degradation of the extracellular matrix is an important step that enables both invasion and metastasis of cancer cells. The proteolytic activity of proteins in the plasminogen activation system (PAS) can degrade ECM either directly or through activation of matrix metalloproteinases (MMPs). PAS proteins include urokinase-type plasminogen activator PLAU (uPA) and its cell surface-associated receptor PLAUR (UPAR) patients (See for instance Andreasen P A, Kjoller L, Christensen L, Duffy M J: The urokinase-type plasminogen activator system in cancer metastasis: a review. Int J Cancer 72:1-22, 1997). PLAU converts inactive plasminogen into its proteolytically active form, serine plasmin. Several colorectal cancer studies show that elevated tissue expression of PLAU is related to increased risk of metastasis and decreased survival, and that these markers are independent prognostic indicators (See for instance Papadopoulou S et al., Significance of urokinase-type plasminogen activator and plasminogen activator inhibitor-1 (PAI-1) expression in human colorectal carcinomas. Tumour Biol 23:170-8, 2002).

Approximately 70% of primary colorectal cancers have loss of heterozygosity (LOH) of chromosomal region 18q (See for instance Fearon E R et al., Identification of a chromosome 18q gene that is altered in colorectal cancers. Science 247:49-56, 1990). This is associated with poor prognosis in colorectal cancer patients. DCC (deleted in colorectal cancer) is a candidate tumor suppressor gene that is present in this region. It encodes several type I transmembrane glycoproteins through alternative splicing. The extracellular domain is very similar to that of the neural cell adhesion molecule (NCAM) family of proteins (See Fearon E R et al., Ibid.), which function in homophilic cell-cell interactions, suggesting that DCC may be involved in cell adhesion. However, more recent evidence indicates that DCC functions as a “dependence receptor” for NTN1 (netrin-1) (See for instance Keino-Masu K et al.: Deleted in Colorectal Cancer (DCC) encodes a netrin receptor. Cell 87:175-85, 1996). NTN1 is a secreted protein that regulates cell migration and outgrowth in the developing nervous system, but it is also found in the colon (See for instance Mazelin L. et al., Netrin-1 controls colorectal tumorigenesis by regulating apoptosis. Nature 431:80-4, 2004). The DCC receptor can promote apoptosis through a caspase-dependent pathway, which is blocked by the presence of the NTN1 ligand. Although the function of DCC is still under investigation, these studies raise the intriguing model that loss of DCC gives cells a selective survival advantage when they stray beyond the effects of the survival ligand secreted by their native tissue. In fact, it was recently shown that inhibition of DCC-induced death in mouse gut leads to tumor formation (See for instance Mazelin L. et al., Ibid). Although it is possible that loss of a gene (or genes) other than DCC may account for the worse prognosis of colorectal cancer patients with 18q LOH, other studies looking directly at loss of DCC expression correlate it with poor prognosis and risk of metastasis (See for instance Gal R. et al., Deleted in colorectal cancer protein expression as a possible predictor of response to adjuvant chemotherapy in colorectal cancer patients. Dis Colon Rectum 47:1216-24, 2004).

CDKN1B (p27/Kip1) is a cyclin-dependent kinase (CDK) inhibitor that inhibits transition from quiescence into a proliferative state by preventing activation of cyclin/CDK complexes, such as cyclin E/CDK2 112. There is also evidence that it plays a role in apoptosis (See for instance Katayose Y et al., Promoting apoptosis: a novel activity associated with the cyclin-dependent kinase inhibitor p27. Cancer Res 57:5441-5, 1997) and inhibition of anchorage-independent growth (See for instance Orend G. et al., Cytoplasmic displacement of cyclin E-cdk2 inhibitors p21Cip1 and p27Kip1 in anchorage-independent cells. Oncogene 16:2575-83, 1998). CDKN1B is deregulated in cancers through accelerated degradation or mislocalization to the cytoplasm. Reduced CDKN1B levels are associated with invasiveness, aggressiveness and poor prognosis in a number of cancer types, including breast, prostate, colon, gastric, lung, and esophageal (See for instance Tsihlias J. et al., The prognostic significance of altered cyclin-dependent kinase inhibitors in human cancer. Annu Rev Med 50:401-23, 1999). Several studies establish that loss or mislocalization of CDKN1B protein is related to worse prognosis in colorectal cancer patients (See for instance Yao J et al., Down-regulation of p27 is a significant predictor of poor overall survival and may facilitate metastasis in colorectal carcinomas. Int J Cancer 89:213-6, 2000).

Thymidylate synthase (TYMS) is a key enzyme in deoxyribonucleotide synthesis and is the primary target of fluorouracil (FU). A recent meta-analysis of 20 studies on the usefulness of TYMS as a prognostic indicator in colorectal cancer concluded that patients with tumors expressing high levels of TYMS appeared to have worse outcomes than those with low levels (See for instance Popat S et al., Thymidylate synthase expression and prognosis in colorectal cancer: a systematic review and meta-analysis. J Clin Oncol 22:529-36, 2004). However, the authors cautioned that disagreement in the literature and potential publication bias warrants further study. In a set of 442 colorectal cancer patients who received surgery only (almost all of whom had stage II disease), high TYMS levels correlated with poor survival (See for instance Edler D et al., Thymidylate synthase expression in colorectal cancer: a prognostic and predictive marker of benefit from adjuvant fluorouracil-based chemotherapy. J Clin Oncol 20:1721-8, 2002).

In response to DNA damage and other stresses, the tumor suppressor TP53 induces growth arrest or apoptosis. Mutations in TP53, most of which lead to elevated basal levels of the protein, are observed in around half of colorectal cancers. A large number of studies have been published on the potential use of TP53 as a molecular marker in colorectal cancer, but results have been mixed. A meta-analysis of these studies concluded that p53 is a marginally significant prognostic indicator (See for instance Petersen S et al., The results of colorectal cancer treatment by p53 status: treatment-specific overview. Dis Colon Rectum 44:322-33; discussion 33-4, 2001).

Similar to CDKN1B, CDKN1A is an inhibitor of cyclin-dependent kinases that restrains progression through the cell cycle. It is inducible by TP53 when a cell is under stress. Some studies have found CDKN1A to be a prognostic indicator, at least in univariate analysis (See for instance Pasz-Walczak G et al., P21 (WAF1) expression in colorectal cancer: correlation with P53 and cyclin D1 expression, clinicopathological parameters and prognosis. Pathol Res Pract 197:683-9, 2001).

ERBB2 and EGFR are growth factor receptor tyrosine kinases that initiate cell survival and proliferation signaling cascades. ERBB2 overepxression has demonstrated prognostic significance in breast and gastric cancers. A few studies indicate that ERBB2 is elevated in a significant percentage of colorectal cancers, as well, and that elevated levels are associated with worse outcome (See for instance Kapitanovic S et al., The expression of p185(HER-2/neu) correlates with the stage of disease and survival in colorectal cancer. Gastroenterology 112:1103-13, 1997). EGFR overexpression has demonstrated prognostic significance in breast and ovarian cancers. A few studies indicate that EGFR is also elevated in a significant number of colorectal cancers, although a prognostic role has not been firmly established (See for instance Mayer A et al., The prognostic significance of proliferating cell nuclear antigen, epidermal growth factor receptor, and mdr gene expression in colorectal cancer. Cancer 71:2454-60, 1993)

In addition to their role as general prognostic factors, several of the above markers may serve the dual purpose of helping to predict drug benefit for agents that are already approved or are under investigation for the treatment of colorectal cancer, such as TYMS (FU), VEGF (bevacizumab/Avastin™), ERBB2 (cetuximab/Erbitux™), and EGFR (erlotinib/Tarceva™).

Method for Defining Panels of Markers

In practice, data may be obtained from a group of subjects. The subjects may be patients who have been tested for the presence or level of certain polypeptides and/or clinicopathological variables (hereafter ‘markers’ or ‘biomarkers’). Such markers and methods of patient extraction are well known to those skilled in the art. A particular set of markers may be relevant to a particular condition or disease. The method is not dependent on the actual markers. The markers discussed in this document are included only for illustration and are not intended to limit the scope of the invention. Examples of such markers and panels of markers are described above in the instant invention and the incorporated references.

Well-known to one of ordinary skill in the art is the collection of patient samples. A preferred embodiment of the instant invention is that the samples come from two or more different sets of patients, one a disease group of interest and the other(s) a control group, which may be healthy or diseased in a different indication than the disease group of interest. For instance, one might want to look at the difference in markers between patients who have had chemotherapy and had a recurrence of cancer within a certain time period and those who had chemotherapy and did not have recurrence of cancer within the same time period to differentiate between the two populations.

When obtaining tumor samples for testing according to the present invention, it is generally preferred that the samples represent or reflect characteristics of a population of patients or samples. It may also be useful to handle and process the samples under conditions and according to techniques common to clinical laboratories. Although the present invention is not intended to be limited to the strategies used for processing tumor samples, we note that, in the field of pathology, it is often common to fix samples in buffered formalin, and then to dehydrate them by immersion in increasing concentrations of ethanol followed by xylene. Samples are then embedded into paraffin, which is then molded into a “paraffin block” that is a standard intermediate in histologic processing of tissue samples. The present inventors have found that many useful antibodies to biomarkers discussed herein display comparable binding regardless of the method of preparation of tumor samples; those of ordinary skill in the art can readily adjust observations to account for differences in preparation procedure.

In preferred embodiments of the invention, large numbers of tissue samples are analyzed simultaneously. In some embodiments, a tissue array is prepared. Tissue arrays may be constructed according to a variety of techniques. According to one procedure, a commercially-available mechanical device (e.g., the manual tissue arrayer MTA1 from Beecher Instruments of Sun Prairie, Wis.) is used to remove an 0.6-micron-diameter, full thickness “core” from a paraffin block (the donor block) prepared from each patient, and to insert the core into a separate paraffin block (the recipient block) in a designated location on a grid. In preferred embodiments, cores from as many as about 400 patients can be inserted into a single recipient block; preferably, core-to-core spacing is approximately 1 mm. The resulting tissue array may be processed into thin sections for staining with interaction partners according to standard methods applicable to paraffin embedded material. Depending upon the thickness of the donor blocks, as well as the dimensions of the clinical material, a single tissue array can yield about 50-150 slides containing >75% relevant tumor material for assessment with interaction partners. Construction of two or more parallel tissue arrays of cores from the same cohort of patient samples can provide relevant tumor material from the same set of patients in duplicate or more. Of course, in some cases, additional samples will be present in one array and not another.

The tumor test samples are assayed by one or more techniques, well-known for those versed in ordinary skill in the art for various polypeptide levels. Briefly, assays are conducted by binding a certain substance with a detectable label to the antibody of the protein in question to be assayed and bringing such in contact with the tumor sample to be assayed. Any available technique may be used to detect binding between an interaction partner and a tumour sample. One powerful and commonly used technique is to have a detectable label associated (directly or indirectly) with the antibody. For example, commonly-used labels that often are associated with antibodies used in binding studies include fluorochrormes, enzymes, gold, iodine, etc. Tissue staining by bound interaction partners is then assessed, preferably by a trained pathologist or cytotechnologist. For example, a scoring system may be utilised to designate whether the antibody to the polypeptide does or does not bind to (e.g., stain) the sample, whether it stains the sample strongly or weakly and/or whether useful information could not be obtained (e.g., because the sample was lost, there was no tumor in the sample or the result was otherwise ambiguous). Those of ordinary skill in the art will recognise that the precise characteristics of the scoring system are not critical to the invention. For example, staining may be assessed qualitatively or quantitatively; more or less subtle gradations of staining may be defined; etc.

It is to be understood that the present invention is not limited to using antibodies or antibody fragments as interaction partners of inventive tumour markers. In particular, the present invention also encompasses the use of synthetic interaction partners that mimic the functions of antibodies. Several approaches to designing and/or identifying antibody mimics have been proposed and demonstrated (e.g., see the reviews by Hsieh-Wilson et al., Acc. Chem. Res. 29:164, 2000 and Peczuh and Hamilton, Chem. Rev. 100:2479, 2000). For example, small molecules that bind protein surfaces in a fashion similar to that of natural proteins have been identified by screening synthetic libraries of small molecules or natural product isolates (e.g., see Gallop et al., J. Med. Chem. 37:1233, 1994; Gordon et al., J. Med. Chem. 37:1385, 1994)

Any available strategy or system may be utilised to detect association between an antibody and its associated polypeptide molecular marker. In certain embodiments, association can be detected by adding a detectable label to the antibody. In other embodiments, association can be detected by using a labeled secondary antibody that associates specifically with the antibody, e.g., as is well known in the art of antigen/antibody detection. The detectable label may be directly detectable or indirectly detectable, e.g., through combined action with one or more additional members of a signal producing system. Examples of directly detectable labels include radioactive, paramagnetic, fluorescent, light scattering, absorptive and calorimetric labels. Examples of indirectly detectable include chemiluminescent labels, e.g., enzymes that are capable of converting a substrate to a chromogenic product such as alkaline phosphatase, horseradish peroxidase and the like.

Once a labeled antibody has bound a tumor marker, the complex may be visualized or detected in a variety of ways, with the particular manner of detection being chosen based on the particular detectable label, where representative detection means include, e.g., scintillation counting, autoradiography, measurement of paramagnetism, fluorescence measurement, light absorption measurement, measurement of light scattering and the like.

In general, association between an antibody and its polypeptide molecular marker may be assayed by contacting the antibody with a tumor sample that includes the marker. Depending upon the nature of the sample, appropriate methods include, but are not limited to, immunohistochemistry (IHC), radioimmunoassay, ELISA, immunoblotting and fluorescence activates cell sorting (FACS). In the case where the polypeptide is to be detected in a tissue sample, e.g., a biopsy sample, IHC is a particularly appropriate detection method. Techniques for obtaining tissue and cell samples and performing IHC and FACS are well known in the art.

In general, the results of such an assay can be presented in any of a variety of formats. The results can be presented in a qualitative fashion. For example, the test report may indicate only whether or not a particular protein biomarker was detected, perhaps also with an indication of the limits of detection. Additionally the test report may indicate the subcellular location of binding, e.g., nuclear versus cytoplasmic and/or the relative levels of binding in these different subcellular locations. The results may be presented in a semi-quantitative fashion. For example, various ranges may be defined and the ranges may be assigned a score (e.g., 0 to 5) that provides a certain degree of quantitative information. Such a score may reflect various factors, e.g., the number of cells in which the tumor marker is detected, the intensity of the signal (which may indicate the level of expression of the tumor marker), etc. The results may be presented in a quantitative fashion, e.g., as a percentage of cells in which the tumor marker is detected, as a concentration, etc. As will be appreciated by one of ordinary skill in the art, the type of output provided by a test will vary depending upon the technical limitations of the test and the biological significance associated with detection of the protein biomarker. For example, in the case of certain protein biomarkers a purely qualitative output (e.g., whether or not the protein is detected at a certain detection level) provides significant information. In other cases a more quantitative output (e.g., a ratio of the level of expression of the protein in two samples) is necessary.

The resulting set of values are put into a database, along with outcome, also called phenotype, information detailing the treatment type, for instance tamoxifen plus chemotherapy, once this is known. Additional patient or tumour test sample details such as patient nodal status, histological grade, cancer stage, the sum total called patient clinicopathological information, are put into the database. The database can be simple as a spreadsheet, i.e. a two-dimensional table of values, with rows being patients and columns being filled with patient marker and other characteristic values.

From this database, a computerized algorithm can first perform pre-processing of the data values. This involves normalisation of the values across the dataset and/or transformation into a different representation for further processing. The dataset is then analysed for missing values. Missing values are either replaced using an imputation algorithm, in a preferred embodiment using KNN or MVC algorithms, or the patient attached to the missing value is excised from the database. If greater than 50% of the other patients have the same missing value then value can be ignored.

Once all missing values have been accounted for, the dataset is split up into three parts: a training set comprising 33-80% of the patients and their associated values, a testing set comprising 10-50% of the patients and their associated values, and a validation set comprising 1-50% of the patients and their associated values. These datasets can be further sub-divided or combined according to algorithmic accuracy. A feature selection algorithm is applied to the training dataset. This feature selection algorithm selects the most relevant marker values and/or patient characteristics. Preferred feature selection algorithms include, but are not limited to, Forward or Backward Floating, SVMs, Markov Blankets, Tree Based Methods with node discarding, Genetic Algorithms, Regression-based methods, kernel-based methods, and filter-based methods.

Feature selection is done in a cross-validated fashion, preferably in a naïve or k-fold fashion, as to not induce bias in the results and is tested with the testing dataset. Cross-validation is one of several approaches to estimating how well the features selected from some training data is going to perform on future as-yet-unseen data and is well-known to the skilled artisan. Cross validation is a model evaluation method that is better than residuals. The problem with residual evaluations is that they do not give an indication of how well the learner will do when it is asked to make new predictions for data it has not already seen. One way to overcome this problem is to not use the entire data set when training a learner. Some of the data is removed before training begins. Then when training is done, the data that was removed can be used to test the performance of the learned model on “new” data.

Once the algorithm has returned a list of selected markers, one can optimize these selected markers by applying a classifier to the training dataset to predict clinical outcome. A cost function that the classifier optimizes is specified according to outcome desired, for instance an area under receiver-operator curve maximising the product of sensitivity and specificity of the selected markers, or positive or negative predictive accuracy. Testing of the classifier is done on the testing dataset in a cross-validated fashion, preferably naïve or k-fold cross-validation. Further detail is given in U.S. patent application Ser. No. 09/611,220, incorporated by reference. Classifiers map input variables, in this case patient marker values, to outcomes of interest, for instance, prediction of stroke sub-type. Preferred classifiers include, but are not limited to, neural networks, Decision Trees, genetic algorithms, SVMs, Regression Trees, Cascade Correlation, Group Method Data Handling (GMDH), Multivariate Adaptive Regression Splines (MARS), Multilinear Interpolation, Radial Basis Functions, Robust Regression, Cascade Correlation+Projection Pursuit, linear regression, Non-linear regression, Polynomial Regression, Regression Trees, Multilinear Interpolation, MARS, Bayes classifiers and networks, and Markov Models, and Kernel Methods.

The classification model is then optimised by for instance combining the model with other models in an ensemble fashion. Preferred methods for classifier optimization include, but are not limited to, boosting, bagging, entropy-based, and voting networks. This classifier is now known as the final predictive model. The predictive model is tested on the validation data set, not used in either feature selection or classification, to obtain an estimate of performance in a similar population.

The predictive model can be translated into a decision tree format for subdividing the patient population and making the decision output of the model easy to understand for the clinician. The marker input values might include a time since symptom onset value and/or a threshold value. Using these marker inputs, the predictive model delivers diagnostic or prognostic output value along with associated error. The instant invention anticipates a kit comprised of reagents, devices and instructions for performing the assays, and a computer software program comprised of the predictive model that interprets the assay values when entered into the predictive model run on a computer. The predictive model receives the marker values via the computer that it resides upon.

Once patients are exhibiting symptoms of cancer, for instance colorectal cancer, a tissue tumor sample is taken from the patient using standard techniques well known to those of ordinary skill in the art and assayed for various tumor markers of cancer by slicing it along its radial axis and placing such slices upon a substrate for molecular analysis by assaying for various molecular markers. Assays can be preformed through immunohistochemistry or through any of the other techniques well known to the skilled artisan. In a preferred embodiment, the assay is in a format that permits multiple markers to be tested from one sample, such as the Aqua Platform™, and/or in a quantitative fashion, defined to within 10% of the actual value and in the most preferred enablement of the instant invention, within 1% of the actual value. The values of the markers in the samples are inputted into the trained, tested, and validated algorithm residing on a computer, which outputs to the user on a display and/or in printed format on paper and/or transmits the information to another display source the result of the algorithm calculations in numerical form, a probability estimate of the clinical diagnosis of the patient. There is an error given to the probability estimate, in a preferred embodiment this error level is a confidence level. The medical worker can then use this diagnosis to help guide treatment of the patient.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses. Marker antibodies or antigens may be incorporated into immunoassay diagnostic kits depending upon which marker autoantibodies or antigens are being measured. A first container may include a composition comprising an antigen or antibody preparation. Both antibody and antigen preparations should preferably be provided in a suitable titrated form, with antigen concentrations and/or antibody titers given for easy reference in quantitative applications.

The kits may also include an immunodetection reagent or label for the detection of specific immunoreaction between the provided antigen and/or antibody, as the case may be, and the diagnostic sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction is detected or quantified by means of detecting or quantifying the label. Immunodetection reagents and processes suitable for application in connection with the novel methods of the present invention are generally well known in the art.

The reagents may also include ancillary agents such as buffering agents and protein stabilizing agents, e.g., polysaccharides and the like. The diagnostic kit may further include where necessary agents for reducing background interference in a test, agents for increasing signal, software and algorithms for combining and interpolating marker values to produce a prediction of clinical outcome of interest, apparatus for conducting a test, calibration curves and charts, standardization curves and charts, and the like.

Various aspects of the invention may be better understood in view of the following detailed descriptions, examples, discussion, and supporting references.

### EXAMPLES

### Example I

### Patients

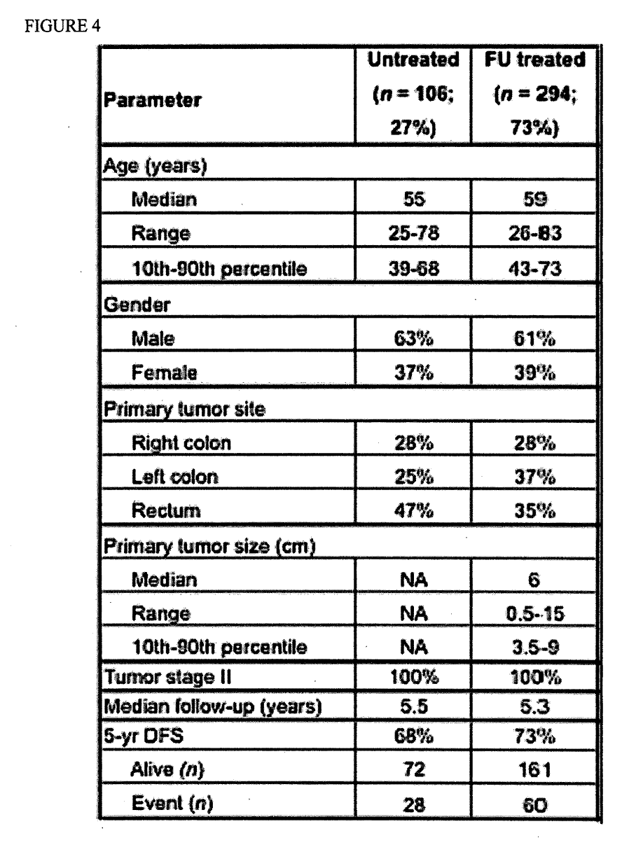

Specimens for two separate Burnham Institute cohorts were obtained from the Yonsei University Department of Pathology, Soeul, Korea, under Institutional Review Board approval (FIG. 4). All patients were stage II as defined by the American Joint Committee on Cancer. Patients in the untreated cohort (no adjuvant chemotherapy) underwent curative surgery between 1986 and 1996. The patients in the treated cohort underwent curative surgery between 1996 and 1999 and then received a FU-based adjuvant chemotherapy regimen.

Assays

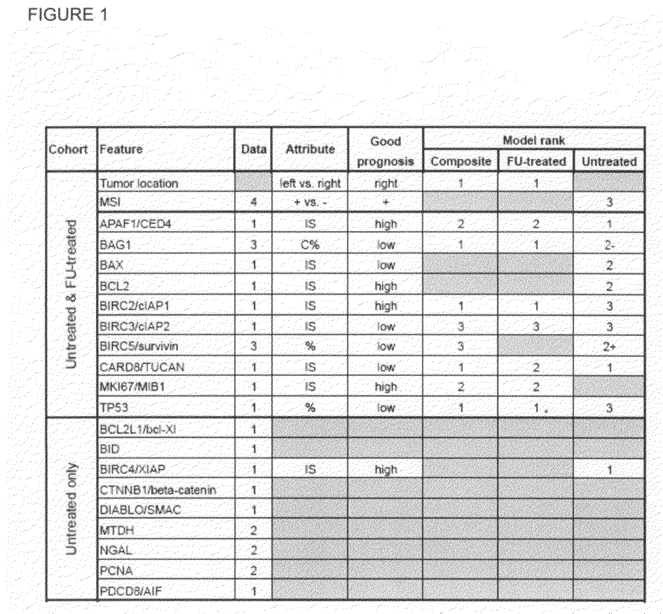

For IHC analyses, formalin-fixed, paraffin-embedded samples were cored and arrayed, and 4-mm sections were stained and scored. FIG. 1 contains a list of the molecular markers assayed. Standard IHC staining methods were used and slides were developed with diaminobenzidine (DAB) See Krajewska et al. Analysis of apoptosis protein expression in early-stage colorectal cancer suggests opportunities for new prognostic biomarkers. Clin Cancer Res 11:5451-61, 2005 for details on the IHC staining methods, as well as a list of the antibodies used.

The percentage of positively stained cells (0-100%) and the intensity of the staining (0, absent; 1, weak; 2, moderate; 3, strong) were determined semi-quantitatively by pathologists. Results from either triplicate or quadruplicate cores were averaged. For some markers, these attributes were determined in both the tumor cells and normal epithelial cells of the same patient, and, for some markers, separate scoring was done for nuclear and cytoplasmic staining (see FIG. 1). Immunoscores were calculated by multiplying the percentage and intensity values, resulting in scores ranging from 0-300. In cases where both normal and tumor cells were assayed, ratios were also calculated. During modeling, all of the various attributes for each marker were treated as continuous variables, and they were also treated as categorical variables based on medians and various other cut-offs.

Microsatellite instability (MSI) analysis was conducted by PCR amplification of six microsatellite markers (five of which are the NCI recommended panels): D2S123, D5S346, D17S250, BAT25, BAT26, and BAT40. Samples in which three or more markers exhibited instability were considered MSI-H (high), patients in which one or two markers exhibited instability were considered MSI-L (low), and samples that exhibited instability in no markers were considered microsatellite stable (MSS). See Krajewska et al. (Ibid.) for additional details.

### Example II

### Methods Providing Input Data to the Mathematical Analysis of the Present Invention

Analyses were conducted in MATLAB version R13 (The Mathworks, Inc., Natick, Mass.) with the Spider package, and in R with the Survival package. During an initial pre-processing stage, the data were coded and characterized, outcome measures were defined, and standard univariate statistical methods were used to identify potentially key markers. Five-year DFS was used as the outcome measure for the development of the models. Specifically, as a measure suitable for supervised machine learning, patients who remained alive and disease-free for at least 5 years were considered survivors, and patients who died or had a recurrence within 4.5 years were considered non-survivors. The 6-month separation between non-survivors and survivors was included to facilitate the machine learning process. Patients who were alive and disease-free, but who were lost to follow-up within five years, were censored and not used for model development. However, they were used in subsequent survival analyses to help assess model performance.

The dataset was then divided into folds for model development employing naïve stratified nested cross-validation to allow robust detection of higher-order correlations in the data. The subjects were divided into five disjoint sets, each forming a naïve test set. The sets were constructed using randomization, but the randomization was applied within stratified subgroups to ensure that the five sub-sets were as fully representative of the complete population of patients as possible accounting for outcome and particular markers that exhibited univariate statistical significance. The complements to each of these five naïve test sets were five training sets. Of the total of 400 patients, there were 271 patients with outcome data satisfying the stated outcome measure constructed from 5-year DFS. The naïve test sets had approximately 55 subjects each, leaving approximately 220 subjects for feature selection and model parameter estimation in each fold.

The overall modeling analysis was conducted in three phases. In the first phase, only the ten IHC markers common to both the untreated and treated cohorts (FIG. 1) were considered for analysis, along with MSI and other standard data, including patient age, patient gender, and tumor location (right colon, left colon, or rectum). Both the untreated and FU-treated cohorts were combined during this first modeling phase, and treatment was made available for inclusion in the model during feature selection.

Kernel partial least squares (KPLS) was selected as the modeling method, and the area under a receiver-operating characteristic (ROC) curve was used as the cost function. The kernel parameters employed were polynomial third-order with the maximum number of latent features set at nine during feature selection. A wrapper-based sequential floating forward feature selection (SFFS) approach was used in conjunction with KPLS to identify high performing sets of features and to estimate model parameters.

The application of these algorithms formed non-linear “maps” of the effects of various factors upon the clinical outcome of interest from the input data, and they were then tested upon previously unseen data using stratified cross-validation to determine the model performance. During the training process each of the five complete training data sets was divided into a subset for model parameter estimation and a corresponding subset for assessing model/feature utility. This process was completed for each of the five training sets, and then the resulting models were applied to their corresponding naïve test set to evaluate the model performance.

After modeling was completed for each of the five data subsets, an “ensemble” model was produced by combining their results. The final ensemble model was then used to assign a prognostic score to each patient with complete data, including both uncensored and censored patients. For the patients who were included in the model development (uncensored with complete outcome data), predictions from the naïve cross-validation were used to assign the scores. For patients who had been excluded from model development due to censored outcome data, the ensemble model was applied to generate the scores. In both cases, the scores for each patient were derived such that the patient's data had never been used in either feature selection or model parameter estimation. The scores essentially ranged from −1 (best prognosis) to +1 (worst prognosis), allowing a threshold (ROC curve operating point) to be selected to alter model sensitivity and specificity. The final “composite” model (trained on both untreated and treated patients) included the 9 parameters with ranked scores under “Composite” in FIG. 1.

Results

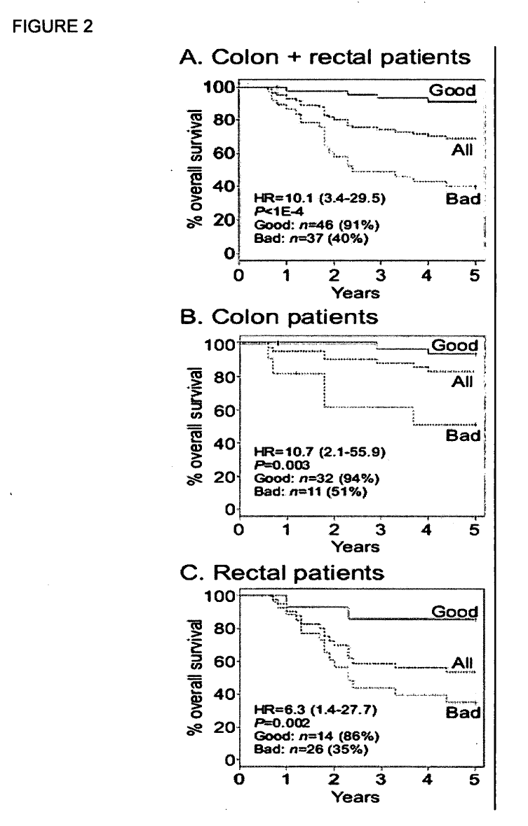

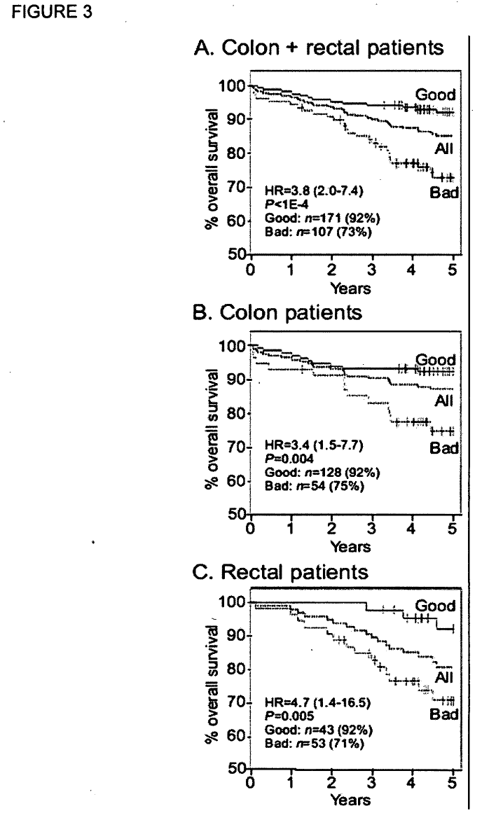

Kaplan-Meier survival and associated Cox proportional hazards analyses were conducted for the two cohorts of patients (untreated and FU-treated), as well as for various subsets of patients, including colon and rectal (FIGS. 2-3) using naïve model score results. Prognostic model score thresholds were chosen to achieve a 5-year OS of ˜92% in the model's “good prognosis” group for each subset. One exception was the untreated rectal cancer subset, which had a relatively low baseline 5-year OS of 53%, where the model's good prognosis group had a 5-year OS of 86%.

The KPLS “composite” model performed extremely well in the untreated cohort of patients. The baseline 5-year OS of all untreated colorectal cancer patients was 68%. The model categorized 55% of these patients into a “good prognosis” group with a 5-year OS of 91%, and 45% of the patients into a “bad prognosis” group with a 5-year OS of 40% (hazard ratio of 10.1) (FIG. 2A). This strong performance was maintained in both the colon and rectal subsets of patients (FIGS. 2B-C). The KPLS “composite” model also performed very well in the FU-treated cohort of patients. The 5-year OS of all treated colorectal cancer patients was 86%. The model categorized 62% of these patients into a “good prognosis” group with a 5-year OS of 92%, and 38% of the patients into a “bad prognosis” group with a 5-year OS of 73% (hazard ratio of 3.8) (FIG. 3A). This strong performance was maintained in both the colon and rectal subsets of patients (FIGS. 3B-C).

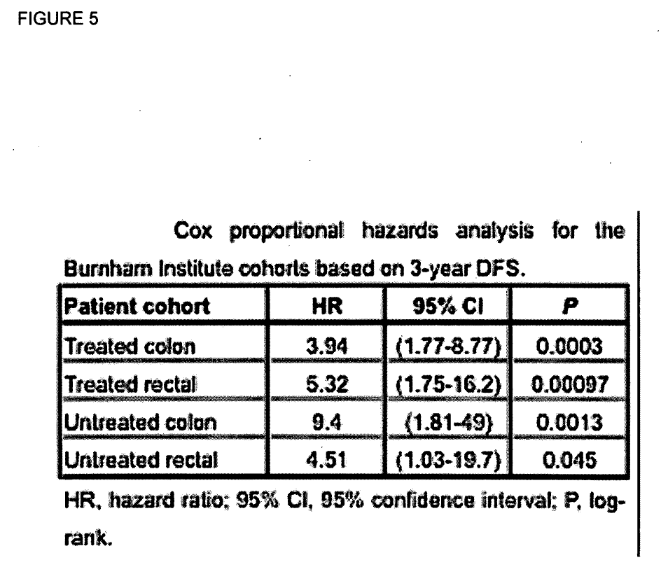

Generally, the good prognosis groups had equivalently good outcomes regardless of whether they were untreated or FU-treated, and the bad prognosis groups in the treated patient cohort had a roughly 30% improved survival relative to the untreated patients (FIGS. 2-3). With the appropriate caveats, this suggests that, in stage II colorectal cancer patients, the model can identify a good prognosis group with high survival and for whom adjuvant FU is unlikely to be beneficial, as well as a bad prognosis group with lower survival and for whom adjuvant FU is likely to show significant benefit. Five-year OS is presented in the survival curves, because, historically, it has been the most widely used metric for analysis of colorectal cancer patients. However, 3-year DFS has been established as a valid surrogate, so we also conducted Cox proportional hazards analysis based on that outcome measure with the “composite” model, and the results were very similar (FIG. 5).

In this study, single markers, like CARD8, as well as additive marker pairs, like CARD8 and BIRC3, performed relatively well in the untreated cohort. However, this is likely an artifact of the relatively small untreated cohort, as none of the individual markers or additive two-marker combinations assessed in the original study in the untreated patients produced statistically significant differentiation of patient outcome in the treated patient cohort (data not shown). However, the fact that our KPLS model worked on both cohorts, maintaining both statistical and clinical significance, indicates a high likelihood that it will generalize to additional patient populations.

Modeling Phase II: Because the first phase of the modeling was performed on both the treated and untreated patient cohorts, it was possible that some of the features in the ensemble model may have been applicable to only one of the two cohorts. Therefore, as a second modeling phase, recursive feature elimination was performed within each naïve set to investigate the applicability of the individual features specifically to the treated cohort, while still allowing for a naïve performance assessment. The objective was to maintain or improve the cross-validated score while potentially reducing the total number of features required for the model. This analysis revealed that, of the 9 parameters in the composite model, all but BIRC5 contributed prognostic relevance to the FU-treated cohort (see “FU-treated” in FIG. 2).

Modeling Phase III: In a third modeling phase, the untreated patient cohort was assessed independently. There were 10 additional IHC markers available for analysis, which were not present in the treated patient cohort, so the complete 20-marker set was assessed (FIG. 2). Due to the relatively low number of patients and comparatively high number of features available in the untreated cohort, a false positive surrogate measure (a null hypothesis set constructed from the markers) was included to improve the robustness of feature selection. This surrogate set for the null hypothesis utilizes marker data that was intentionally randomized with respect to outcome (by randomizing row order in the data spreadsheet) so that the original joint marker distributional characteristics were maintained, but a relationship with respect to outcome was broken. Fifty features were selected randomly to be included in the null surrogate set. After being randomized with respect to row order, they were appended to the original feature set.

During the joint feature selection and model parameter estimation process, marker selection sets were allowed to include features from the null surrogate set. When this occurred, it indicated that the information in the markers assessed for inclusion in the growing model marker set were not superior to markers with a known random outcome. In most cases, a null surrogate was added only when the number of features already in a model (model order) was high, and once the null surrogate feature was added, it or another null surrogate feature were maintained during subsequent SFFS epochs for that model marker set progression. However, in some cases, the surrogate marker was discarded in a subsequent SFFS epoch, model order was decreased, and only valid features were included thereafter in the model. During post-analysis, the presence of a known false-positive feature was used as a measure to deem those marker sets as over-determined and to be flagged for exclusion from analysis. The cross-validation score for the marker set was then used with this exclusion flag to examine the progression of the marker feature sets to identify those feature sets to include in an ensemble model. A ranked feature list from this analysis is presented under “Untreated” in FIG. 2.

In accordance with these and still other insights obtained by the building, and the exercise, of the diagnostic test in accordance with the present invention, the invention should be broadly defined by the following claims.

We claim:

1. A method of predicting response to adjuvant therapy or predicting disease progression in colorectal cancer comprising:
obtaining a colorectal cancer test sample from a subject;
obtaining clinicopathological data from said colorectal cancer test sample;
analyzing the obtained colorectal cancer test sample for presence or amount of (1) one or more molecular markers of loss of function of mismatch repair proteins, one or more molecular markers of apoptosis, one or more proliferation markers, and one or more tumor suppression molecular markers; (2) one or more additional molecular markers both proteomic and non-proteomic that are indicative of colorectal cancer disease processes consisting essentially of the group comprised of: angiogenesis, catenin/cadherin proliferation/differentiation, cell cycle processes, cell surface processes, cell-cell interaction, cell migration, centrosomal processes, cellular adhesion, cellular proliferation, cellular metastasis, invasion, cytoskeletal processes, ERBB2 interactions, growth factors and receptors, membrane/integrin/signal transduction, metastasis, oncogenes, proliferation, proliferation oncogenes, signal transduction, surface antigens and transcription factor molecular markers; and then
correlating (1) the presence or amount of said molecular markers and, with (2), clinicopathological data from said tissue sample other than the molecular markers of colorectal cancer disease processes, in order to deduce a probability of response to chemotherapy or future risk of disease progression in colorectal cancer for the subject.

2. The method according to claim 1
wherein the correlating is in order to deduce a probability of response to a specific adjuvant therapy which is a specific chemotherapy drawn from the group consisting of Irinotecan, Leucovorin, and/or 5-Fluorouracil; and/or which is a specific platinum therapy drawn from the group consisting of satraplatin, oxliplatin, carboplatin, or cisplatin, and/or which is a specific targeted therapy drawn from the group consisting of bevacizumab, panitumumab, or cetuximab.

3. The method according to claim 1 wherein the correlating comprises:
determining the expression levels or mass spectrometry peak levels or mass-to-charge ratio(s) of one or more proteomic marker(s) and the numerical quantity of one or more clinicopathological marker(s) from colorectal cancer test sample excised from a patient population P1 before therapeutic treatment, clinical outcome C1 after a certain time period on said patient population P1 not known in advance; and
comparing said determined levels and numerical values to another set of expression levels or mass spectrometry peak levels or mass-to-charge ratio(s) of one or more proteomic marker(s) and the numerical quantity of one or more clinicopathological marker(s) from colorectal cancer test sample excised from a separate patient population P2 before therapeutic treatment, clinical outcome C2 after said certain time period on said patient population P2 known in advance;
wherein the clinical outcome C1 and C2 is drawn from the group consisting essentially of: colorectal cancer disease diagnosis, disease prognosis, or treatment outcome or a combination of any two, three or four of these outcomes; and
training an algorithm to identify characteristic expression levels or mass spectrometry peak levels or mass-to-charge ratio(s) of one or more proteomic marker(s) and numerical quantity(ies) of one or more clinicopathological marker(s) between said patient population P1 and patient population P2 which correlate to clinical outcome C1 and clinical outcome C2, respectively.

4. The method according to claim 1 wherein the training of the algorithm on characteristic protein levels or patterns of differences comprises the steps of
obtaining numerous examples of (I) said expression levels or mass spectrometry peak levels or mass-to-charge ratio(s) of one or more proteomic marker(s) and numerical quantity(ies) of one or more clinicopathological marker(s) data, and (ii) historical clinical results corresponding to this proteomic marker(s) and clinicopathological marker(s) data;
constructing an algorithm suitable to map (I) said characteristic proteomic and said clinicopathological marker(s) data values as inputs to the algorithm, to (ii) the historical clinical results as outputs of the algorithm;
exercising the constructed algorithm to so map (I) the said protein expression levels or mass spectrometry peak or mass-to-charge ratio(s) and clinicopathological marker(s) values as inputs to (ii) the historical clinical results as outputs; and
conducting an automated procedure to vary the mapping function inputs to outputs, of the constructed and exercised algorithm in order that, by minimizing an error measure of the mapping function, a more optimal algorithm mapping architecture is realized;
wherein realization of the more optimal algorithm mapping architecture, also known as feature selection, means that any irrelevant inputs are effectively excised, meaning that the more optimally mapping algorithm will substantially ignore specific proteomic marker(s) and specific clinicopathological marker(s) values that are irrelevant to output clinical results; and
wherein realization of the more optimal algorithm mapping architecture, also known as feature selection, also means that any relevant inputs are effectively identified, making that the more optimally mapping algorithm will serve to identify, and use, those input protein expression levels or mass spectrometry peak or mass-to-charge ratio(s) and said clinicopathological marker(s) values that are relevant, in combination, to output clinical results that would result in a clinical detection of disease, disease diagnosis, disease prognosis, or treatment outcome or a combination of any two, three or four of these actions.

5. The method according to claim 4
wherein the constructed algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

6. The method according to claim 4
wherein the feature selection process employs an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

7. The method according to claim 4 further comprising:
training a tree algorithm is trained to reproduce the performance of another machine-learning classifier or regression by enumerating the input space of said classifier or regression to form a plurality of training examples sufficient (1) to span the input space of said classifier or regression and (2) train the tree to emulate the performance of said classifier or regression.

8. The method according to claim 1
wherein the correlating so as to predict the response to adjuvant therapy or disease progression is particularly so as to predict the response to chemotherapy or tumor aggressiveness respectively;
and wherein the method further comprises:
diagnosing colorectal cancer in a patient by taking a biopsy of colorectal cancer tissue and identifying that said biopsy is wholly or partially malignant;
identifying clinicopathological values associated with said malignant biopsy;
analyzing said malignant tissue for the proteomic markers APAF1/CED4, BAG1, BIRC2/cIAP1, BIRC3/cIAP2, BIRC4/XIAP, CARD8/TUCAN, MKI-67/MIB1, and TP-53, and one or more additional proteomic markers;
evaluating the patient's prediction of response of said tumor to said therapy or evaluated risk of disease progression, respectively from said measured levels of proteomic markers and clinicopathological values; and
administering chemotherapy or other therapy as appropriate to the evaluated prediction of response of said tumor to said therapy or evaluated risk of disease progression, respectively.

9. The method according to claim 8
wherein the one or more additional markers includes, in addition to markers APAF1/CED4, BAG1, BIRC2/cIAP1, BIRC3/cIAP2, BIRC4/XIAP, CARD8/TUCAN, MKI-67/MIB1, and TP-53, the proteomic markers VEGF, PLAU, PLAUR, DCC, CDKN1A, CDKN1B/p27, TYMS, ERBB2, EGFR, PTGS2, CTNNB1, SFRP4, and nuclear MLH1 and MSH2.

10. The method according to claim 8
wherein the one or more additional markers includes, in addition to markers APAF1/CED4, BAG1, BIRC2/cIAP1, BIRC3/cIAP2, BIRC4/XIAP, CARD8/TUCAN, MKI-67/MIB1, and TP-53, a proteomic marker of receptor-based growth/death signaling.

11. The method of claim 10
wherein the analyzing is of one or more additional markers of colorectal cancer disease processes in addition to one or more molecular markers of apoptosis, one or more growth factor receptor markers, and one or more cell adhesion, metastasis, angiogenesis, nucleotide biosynthesis, or invasion molecular markers is of one or more markers selected from the group consisting of two or more of the following: CD105, TGFbeta, NRP, VEGF, DNA ploidy, Bcl-2, BAX, SFRP4, or markers related thereto.

12. The method of claim 10
wherein the correlating is further so as to determine colorectal cancer treatment response or prognostic outcome; and
wherein the correlating is performed in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

13. The method of claim 12
wherein the correlating so as to further determine treatment outcome is, in addition to prediction of response to chemotherapy, expanded to prediction of response to platinum therapy and targeted therapy.

14. The method of claim 13
wherein the correlating is of clinicopathological data selected from a group consisting of tumor nodal status, tumor grade, tumor size, tumor location, patient age, previous personal and/or familial history of colorectal cancer, previous personal and/or familial history of response to colorectal cancer therapy, and microsatellite instability analysis.

15. The method of claim 1
wherein the analyzing is of both proteomic and clinicopathological markers; and
wherein the correlating is further so as to a clinical detection of disease, disease diagnosis, disease prognosis, or treatment outcome or a combination of any two, three or four of these actions.

16. The method of claim 1
wherein the obtaining of the test sample from the subject is of a test sample selected from the group consisting of fixed, paraffin-embedded tissue, colorectal cancer tissue biopsy, tissue microarray, fresh tumor tissue, fine needle aspirates, peritoneal fluid, ductal lavage and pleural fluid or a derivative thereof.

17. The method of claim 1
wherein the obtaining of the test sample from the subject before treatment of symptoms by a specific therapy; and
wherein the correlating is between (1) proteomic and clinicopathological marker values, and (2) the probability of present or future risk of a colorectal cancer progression for the subject or treatment outcome for said specific therapy, for a time period measured from the obtaining of said test sample chosen from the group consisting essentially of: 6, 12, 18, 24, 36, 60, 84, 120, or 180 months.

18. The method of claim 1
wherein the correlating is in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

19. The method of claim 1
wherein the molecular markers of loss of function of mismatch repair proteins are MLH1 and MSH2, the molecular markers of apoptosis are APAF1, BAG1, BIRC2, BIRC4, and TUCAN, the molecular marker of proliferation is MKI-67, and the tumor suppression molecular marker is TP-53;
wherein the additional one or more molecular marker(s) is selected from the group consisting of essentially: VEGF, EGFR, CDKN1B, SFRP4, PLAU, or TYMS;
wherein the correlating is by usage of a trained kernel partial least squares algorithm; and
wherein the prediction is of outcome of chemotherapy for colorectal cancer.

20. The method of claim 19
wherein the additional one or more molecular marker(s) is TYMS; and
the chemotherapy is 5-fluorouracil therapy.

21. The method of claim 1
wherein the molecular markers of loss of function of mismatch repair proteins are MLH1 and MSH2, the molecular markers of apoptosis are APAF1, BAG1, BIRC2, BIRC4, and TUCAN, the molecular marker of proliferation is MKI-67, and the tumor suppression molecular marker is TP-53;
wherein the additional one or more molecular marker(s) is selected from the group consisting of essentially: VEGF, EGFR, CDKN1B, SFRP4, PLAU, or TYMS;
wherein the correlating is by usage of a trained kernel partial least squares algorithm; and
wherein the prediction is of risk of colorectal cancer progression.

22. The method of claim 1 wherein the molecular markers of loss of function of mismatch repair proteins comprise:
MLH1 and MSH2′
wherein the molecular markers of apoptosis comprise:
APAF1, BAG1, BIRC2, BIRC4, and TUCAN;
wherein the molecular marker of proliferation comprises:
MKI-67;
and wherein the tumor suppression molecular marker comprises:
TP-53;
wherein the additional one or more molecular marker(s) is selected from the group consisting of essentially: VEGF, EGFR, CDKN1B, SFRP4, PLAU, or TYMS;
wherein the clinicopathological data is one or more datum values selected from the group consisting essentially of: tumor size, tumor location, nodal status, and stage;
wherein the correlating is by usage of a trained kernel partial least squares algorithm; and
wherein the prediction is risk of colorectal cancer progression.

23. The method of claim 22
wherein the additional one or more molecular marker(s) is CDKN1B; and
wherein the prediction is of risk of colorectal cancer progression as given by a likelihood score derived from using Kaplan-Meier survival curves.

24. A kit comprising:
a panel of antibodies whose binding with colorectal cancer tumor samples has been correlated with colorectal cancer treatment outcome or patient prognosis;
reagents to assist said antibodies with binding to tumor samples; and
a computer algorithm, residing on a computer, calculating in consideration of analyzed antibodies, interpolates from the aggregation of all binding values upon the colorectal cancer tumor sample the prediction of treatment outcome for a specific treatment for colorectal cancer or future risk of colorectal cancer progression for the subject.

25. The kit according to claim 24 wherein the panel of antibodies comprises:
a poly- or monoclonal antibody specific for an individual protein or protein fragment and that binds one of said antibodies correlated with colorectal cancer treatment outcome or patient prognosis.

26. The kit according to claim 24 wherein the device comprises:
a number of immunohistochemistry assays equal to the number of antibodies.

27. The kit according to claim 24 wherein the antibodies comprise:
antibodies correlated with colorectal cancer treatment outcome and the computer algorithm is an algorithm using kernel partial least squares.

28. The kit according to claim 27 wherein the antibodies comprise:
antibodies specific to MLH1, MSH2, APAF1, BAG1, BIRC2, BIRC4, TUCAN, MKI-67, and TP-53.

29. The kit according to claim 27
wherein the treatment outcome is response to chemotherapy, platinum therapy or targeted therapy.

30. The kit according to claim 27 wherein the antibodies comprise:
antibodies correlated with colorectal cancer progression and the computer algorithm is an algorithm using kernel partial least squares.

31. The kit according to claim 27
wherein the antibodies are antibodies specific MLH1, MSH2, APAF1, BAG1, BIRC2, BIRC4, TUCAN, MKI-67, and TP-53.

32. The kit according to claim 25 wherein the antibodies comprise:
antibodies specific to MLH1, MSH2, APAF1, BAG1, BIRC2, BIRC4, TUCAN, MKI-67, and TP-53; with one or more additional markers selected from the group consisting of VEGF, EGFR, CDKN1B, SFRP4, PLAU, or TYMS; and with one or more additional markers selected from the group consisting of CD105, TGFbeta, NRP, VEGF, DNA ploidy, Bcl-2, BAX, SFRP4, or markers related thereto.
